(19) United States
(12) Patent Application Publication
(10) Pub. No.: US 2008/0089868 A1
(43) Pub. Date: Apr. 17, 2008

(54) Retinal stem cell compositions and methods for preparing and using same

(75) Inventors: Michael Ezra Zuber, Manlius, NY (US); Andrea Sophia Viczian, Manlius, NY (US)

Correspondence Address: HESLIN ROTHENBERG FARLEY & MESITI PC, 5 COLUMBIA CIRCLE, ALBANY, NY, 12203, US

(73) Assignee: The Research Foundation of State University of New York, Albany, NY (US)

(21) Appl. No.: 11/796,171

(22) Filed: Apr. 27, 2007

Related U.S. Application Data
(60) Provisional application No. 60/795,404, filed on Apr. 27, 2006.

(51) Int. Cl. A61K 35/12; A61P 27/02; C12N 5/00; C12N 5/08

(52) U.S. Cl. 424/93.7; 435/366; 435/371; 435/375

(57) ABSTRACT

Provided are cell compositions including non-retinal cell types that have been reprogrammed to form retinal stem cells, and methods for producing and using same. Such reprogrammed cells can be used to replace one or more retinal cell types that have been lost due to damage and/or disease and are thus useful in treating or preventing visual impairment.

### CROSS-REFERENCE TO RELATED APPLICATIONS

This application claims priority from U.S. Provisional Application No. 60/795,404, filed Apr. 27, 2006, which is hereby incorporated by reference in its entirety.

### STATEMENT OF RIGHTS UNDER FEDERALLY SPONSORED RESEARCH

This invention was made with government support under grant number 5R01EY015748-02 entitled “Retinal Stem Cell Culture and Characterization” awarded by the National Eye Institute of U.S. National Institutes of Health. Accordingly, the government has certain rights in the invention.

### FIELD OF THE INVENTION

The present invention relates to retinal stem cell compositions and methods for reprogramming non-retinal cells to retinal stem progenitor cells. Such reprogrammed cells can be used to replace lost retinal cells and thus be used as a method of treating or preventing visual impairment caused by the loss of one or more retinal cell types.

### BACKGROUND OF THE INVENTION

Nearly 10 million Americans are blind or suffer visual impairment due to glaucoma, retinitis pigmentosa, age-related macular degeneration and diabetic retinopathies. These diseases are all due to the loss of one or more retinal cell type and according to the most recent statistics represent 36% of the existing cases of legal blindness in the United States. Every year an additional 230,000 patients are diagnosed with these diseases. Current treatments can slow disease progression, but cannot replace lost retinal cells.

In addition to disease, physical damage to retinal cells may also occur through retinal detachment or other trauma to the eye. The therapeutic strategies for treating loss of vision caused by retinal cell damage vary, buy they are all directed to controlling the illness causing the damage, rather than reversing the damage caused by an illness by restoring or regenerating retinal cells.

Retinal cells are derived from the ectodermal germ layer. A homogenous collection of neuralized ectodermal (neuroectodermal) cells becomes increasingly lineage-restricted in response to extrinsic factors in the local cellular environment thereby generating retinal progenitor cells. In tissues other than the eye, stem cells are used as a source for alternative treatments of disease or injury to tissues. Stem cells are undifferentiated cells that exist in many tissues of embryos and adult mammals. In adults, specialized stem cells in individual tissue are the source of new cells that replace cells lost through cell death due to natural attrition, disease, or injury. Stem cells are ideal for use in tissue replacement therapies. They are multipotent, self-renewing, and can differentiate into cell types of their tissue of origin.

Stem cells are capable of producing either new stem cells or cells called progenitor cells that differentiate to produce the specialized cells found in mammalian organs. In contrast to progenitor cells, stem cells never terminally differentiate. Because retinal stem cells are restricted in their potential (i.e., they only give rise to the cell types found in the eye) they provide an excellent option for replacing cells lost by retinal injury, diseases, or other factors causing visual impairment.

The discovery of human retinal stem cells in the adult eye prompted isolation of these cells from donor tissues to serve as a valuable source of retinal stem cells for transplantation. Adult human retinal stem cells isolated from cadavers grow well in culture, and when induced to differentiate they express markers for mature retinal cell types in vitro. Unfortunately, when transplanted to even the permissive environment of the embryonic mammalian eye, they differentiate into only three of the seven retinal cell types, suggesting restricted fates and a loss in multipotency. Despite their obvious potential, endogenous human adult retinal stem cells do not repair the damaged retina. In addition, as with other transplantation therapies, host rejection is a continuing problem.

Thus, although retinal stem and progenitor cells provide an important opportunity for treating retinal injuries and degenerations, to be used successfully in cell replacement therapies a plentiful source of these cells must be identified. Previous, but unsuccessful, studies have attempted to convert pluripotent embryonic stem cells directly into retinal progenitors. Embryonic stem cells can form tissues from all three germ layers (endoderm, mesoderm, as well as ectoderm), possibly explaining the small, limited number of retinal cells and retinal cell fates generated in these previous experiments. Neuralization of ectoderm alone is not sufficient to generate only retinal progenitors since neuroectoderm also differentiates into other anterior neural structures (e.g., brain tissues).

Accordingly, in view of the deficiencies attendant with the prior art cell compositions and methods, it would be desirable to develop a reliable source of unlimited numbers of retinal stem cells for transplantation, which are capable of differentiating into all of the various retinal cell types.

### SUMMARY OF THE INVENTION

It is an object of the present invention to provide a unique, alternative approach for generating large numbers of reliable multipotent retinal stem/progenitor cells that are not restricted in cell fate and that are capable of differentiating into all of the various retinal cell types.

It is another object of the present invention to convert (i.e., reprogram) plentiful non-retinal cell types to retinal stem/progenitor cells.

It is still another object of the present invention to treat or prevent a variety of visual impairment disorders related to the loss of one or more retinal cell type by repopulating the retinal cells using the non-retinal cells that have been reprogrammed to retinal stem/progenitor cells.

Accordingly, in one aspect the invention provides compositions of retinal stem cells, which include a population of reprogrammed non-retinal cell types.

In another aspect, the invention provides compositions of non-retinal cells that have been reprogrammed by genetically altering the cells to express or over express a gene set encoding the eye field transcription factors necessary to induce the formation of ectopic eyes in vivo and which reprogram non-retinal cells to retinal stem/progenitor cells.

In yet another aspect, the invention provides compositions of non-retinal cells that have been reprogrammed by externally applying one or more secreted activators or inhibitors of a signaling pathway involved in retinal stem cell formation or causing them to express or over-express one or more secreted activators or inhibitors of a signaling pathway involved in retinal stem cell formation.

In another aspect, the invention provides pharmaceutical compositions that include a therapeutically effective amount of the retinal stem/progenitor cell compositions disclosed herein along with a pharmaceutically acceptable diluent, excipient, or carrier.

In another aspect, the invention provides methods for treating or preventing visual impairment by administering a therapeutically effective amount of one of the compositions disclosed herein to a subject in need thereof.

In yet another aspect, the invention provides methods of reprogramming a population of non-retinal cells by genetically altering the cells to express or over express a gene set encoding the eye field transcription factors necessary to induce the formation of ectopic eyes in vivo and which reprogram non-retinal cells to retinal stem/progenitor cells.

In still another aspect, the invention provides methods for reprogramming a population of non-retinal stem cells by externally applying one or more secreted activators or inhibitors of a signaling pathway involved in retinal stem cell formation or causing the cells to express or over-express one or more secreted activators or inhibitors of a signaling pathway involved in retinal stem cell formation.

In another aspect, the invention provides methods of reprogramming embryonic stem cells by exposing the embryonic stem cells to factors causing them to differentiate into ectodermal cells, and then exposing the ectodermal cells to one or more secreted activators or inhibitors of a signaling pathway involved in retinal stem cell formation.

In yet another aspect, the invention provides methods of repopulating one or more retinal cell types by providing a population having one or more non-retinal cell types, causing the cells to express or over-express one or more secreted activator or inhibitor of a signaling pathway involved in retinal stem cell formation, thereby effectively reprogramming the non-retinal cell into a retinal stem cell, and injecting the reprogrammed non-retinal cell (i.e., the retinal stem cell) into the retina of a subject in need thereof.

### BRIEF DESCRIPTION OF THE DRAWINGS

The present invention will be better understood from the detailed description given herein below and the accompanying drawings which are given by way of illustration only, and thus are not a limitative of the present invention and wherein:

FIG. 1: A schematic of the Animal Cap Transplant Assay as further discussed in the Detailed Description: (A) GFP and/or EFTF RNAs are injected into both blastomeres of two-cell stage Xenopus embryos and allowed to grow in 0.4×MMR and 6% ficoll at 14° C. overnight; (B) Stage 15 (host) embryos are placed in a surgical dish in 0.7×MMR and gentamicin, the vitellin membrane is removed using #5 forceps and one eye field is removed using the Gastromaster with a 13 micron tip, bent to a width of 200 μm.

FIG. 2: Images of reprogrammed ectoderm cells using eye field transcription factors. EFTFs reprogram ectoderm to retinal stem cells that form eyes when transplanted into the embryonic eye field: (A-C) Lateral and dorsal views of stage 42 (A, B) and 46 (C) embryos in which the left eye field has been replaced with an EFTF-cap. GFP fluorescence (B) demonstrates the transplanted eye (and some of the skin ectoderm) originate from the transplanted EFTF-cap; (D-F) Control embryo with GFP-cap transplant. No eye forms, (E), and GFP-cap cells are detected only in the skin ectoderm in both whole mount (D) and cryostat sectioned (F) embryos. Arrowheads in (D) are background fluorescence); (G) When only ½ of the eye field is removed and a partial eye forms (arrowhead), the GFP-cap cells do not contribute to the eye; (H) The light-induced (20 ms) ERG response of the induced eye (IE) is indistinguishable in time course and intensity from the control eye ERG; (I and K) Cryostat section of induced eye stained by in situ hybridization for the RGC-specific marker (hermes) and by immunocytochemistry for the rod photoreceptor marker (opsin), demonstrating the presence of ganglion cell and outer nuclear layers. (This section does not pass through the lens, hence the RGC layer appears as a donut rather than a croissant.) (I-K) Black and white lines demarcate the two plexiform layers of the retina, revealing the characteristic tri-layered structure observed in the normal eye: outer nuclear layer (ONL), inner nuclear layer (INL) and ganglion cell layer (GCL). (K) An EFTF-cap containing embryo was injected with BrdU for 1 hour, fixed, sectioned and stained using anti-BrdU antibodies. BrdU is incorporated into the DNA of proliferating cells during S phase. BrdU is observed in the induced eye at the periphery of the ciliary marginal zones, CMZ, the RS cells niche. (L) The magnitude of the b-wave for the two induced eyes tested were a function of flash intensity, saturating, well fit to Michaelis-Menton functions and similar to the response of a control eye at both 520 and 650 nm.

FIG. 3: Images of eye tissue transformed from noggin expressing ectoderm. Cryostat sections through a stage 47 embryo whose eye field at stage 15 was replaced with primitive ectoderm misexpressing (i.e., expressing exogenous protein or over-expressing endogenous protein) and the tracer GFP RNA: (A) Bright field image of the retina overlayed with the fluorescent image (B) magnified 20 times shows pan GFP expression throughout the retina. Arrow points to GFP expression in retinal ganglion cell axons exiting the eye. Arrowheads point to GFP expression in the peripheral region of the retina, which contains the retinal stem cells. Dashed box in (B) shows the region magnified at 40×, which are panels (C) and (D); (C-D) Arrowheads point to area of the retina containing retinal stem cells. Because these cells are GFP positive, they are clearly a contribution of the transplanted tissue. This is evidence that noggin is able to transform ectoderm into retinal stem/progenitor cells.

FIG. 4: Schematic drawing of the reprogramming of embryonic stem cells to retinal stem cells. Embryonic stem (ES) cells, converted to ectoderm-like precursors (EPL) cells, are grown in culture and biased toward a retinal progenitor cell (RPC) fate using extrinsic factors such as noggin individually and in combination with one or more of chordin, cerberus and/or TGF-β3 and conditioned media (MEDII). Retinal stem/progenitor cells will glow green for subsequent purification.

### DETAILED DESCRIPTION OF THE INVENTION

The present invention is based in part on the surprising discovery that non-retinal cells can be reprogrammed to retinal stem cells, which unexpectedly differentiate into all of the various retinal cell types. Thus, the reprogrammed cells can be used to repopulate one or more retinal cell types that have been lost due to disease or injury.

Accordingly, the present invention provides compositions and pharmaceutical formulations containing a population of non-retinal cells that have been reprogrammed to retinal stem cells for use in methods directed to treating subjects suffering from various visual impairment disorders.

The present invention also provides methods for reprogramming non-retinal cells to retinal stem cells, and methods of using same to treat or prevent various visual impairment disorders.

The features and other details of the invention will now be more particularly described with references to the accompanying drawings, examples and claims. Certain terms are defined throughout the specification. Unless otherwise defined, all technical and scientific terms used herein have the same meaning as commonly understood by one of ordinary skill in the art to which this invention pertains. In some cases, terms with commonly understood meanings are defined herein for clarity and/or for ready reference, and the inclusion of such definitions herein should not necessarily be construed to represent a substantial difference over the definition of the term as generally understood in the art. Furthermore, as used herein and in the appended claims, the singular forms include plural referents unless the context clearly dictates otherwise. Thus, for example, reference to “a secreted inhibitor or activator of a signaling pathway” includes one or more of such activators or inhibitors, as would be known to those skilled in the art.

Retinal Stem Cell Compositions

One aspect of the present invention provides a composition including a population of retinal stem cells, the retinal stem cells being comprised of a population of reprogrammed non-retinal cell types. For the purpose of this application the term “reprogramming” or “reprogrammed” can be broadly defined and encompasses the conversion of one cell type into another. For instance, in the context of the present invention, cells that would normally form skin cells are reprogrammed to form cells that differentiate into various retinal cell types.

In one embodiment, the population of retinal stem cells can be derived from a mammal, and more specifically, a human. The term “mammal” or “mammalian” is used in its dictionary sense. The term “mammal” includes, for example, mice, hamsters, rats, cows, sheep, pigs, goats, and horses, monkeys, dogs (e.g., Canis familiaris), cats, rabbits, guinea pigs, and primates, including humans. Such retinal stem cells are capable of producing retinal progenitor cells, retinal cells, and adult retinal stem cells or other appropriate cell types. In one embodiment, the retinal cells can be selected from one or more types of cells located in the retina. These retinal cell types include, for example, rod cells, cone cells, bipolar cells, amacrine cells, retinal ganglion cells, retinal pigment epithelial cells, Mueller cells, and horizontal cells.

In one embodiment, the non-retinal cell types can be selected from, but not limited to, ectodermal cells. More specifically, the ectodermal cells can be epidermal stem cells. In one embodiment, the epidermal stem cells can be reprogrammed embryonic stem cells or cells harvested from a patient's skin. If the non-retinal cells types are harvested from a patient's own skill, the cells are reprogrammed and the cells are used to treat the same patient, that patient acts as an autologous donor.

Further, the non-retinal cell types can be reprogrammed with a gene set containing an eye-field transcription factor (“EFTF”) cocktail (or “EFTFs”). As used herein, the term “eye-field” consists of embryonic retinal stem cells that generate all the retinal cells of the adult eye. As used herein, the term “eye-field transcription factor cocktail” includes, but is not limited to, a cocktail (i.e., combination) of nucleic acid sequences encoding Otx2; ET; Rx1; Pax6; Six3; tll; Optx2; and other transcription factors other orthologs thereof. The corresponding sequence and structures of these transcription factors are known to those skilled in the art and are not reproduced herein. Accession numbers for these factors are provided in the Material and Methods portion of the instant specification.

In another embodiment, the non-retinal cell types can be reprogrammed by externally applying at least one secreted activator or inhibitor of a signaling pathway involved in retinal stem cell formation or causing the non-retinal cells to express or over express at least one secreted activator or inhibitor of a signaling pathway involved in retinal stem cell formation. The signaling pathway can be selected from one or more of, for example, hedgehog (Hh), wingless (Wnt), transforming growth factor-β (TGF-β), bone morphogenic protein (BMP), insulin growth factor (IGF), fibroblast growth factor (FGF), among other signaling pathways. In one embodiment, the activator or inhibitor can be an antagonist of BMP. More specifically, the antagonist of BMP can be selected from one or more of fetuin, noggin, chordin, gremlin, follistatin, Cerberus, amnionless, DAN, the ecto domain of the BMP receptor protein BMRIA, or other appropriate antagonists of BMP. In one embodiment, the BMP antagonist is noggin. In another embodiment, the activator or inhibitor can be TGF-β signaling pathway. In one embodiment, the secreted molecule of the TGF-β pathway can include, but is not limited to, nodal.

In another aspect, the invention provides compositions including a population of non-retinal cell types that have been transfected with a gene set containing EFTFs. In one embodiment, the EFTFs include, but are not limited to, nucleic acid sequences encoding Otx2, ET, Rx1, Pax6 Six3, tll, Optx2, or orthologs thereof.

In another aspect, the invention provides compositions including a population of non-retinal cell types that have been externally treated or transfected with at least one secreted activator or inhibitor of a signaling pathway involved in retinal stem cell formation. Non-retinal cells types may be caused to express or over express at least one secreted activator or inhibitor of a signaling pathway involved in retinal stem cell formation. In one embodiment, the signaling pathway can be selected from one or more of, for example, hedgehog (Hh), wingless (Wnt), transforming growth factor-β (TGF-β), bone morphogenic protein (BMP), insulin growth factor (IGF), fibroblast growth factor (FGF), among other signaling pathways. In another embodiment, the activator or inhibitor can be an antagonist of BMP. More specifically, the antagonist of BMP can be selected from one or more of fetuin, noggin, chordin, gremlin, follistatin, Cerberus, amnionless, DAN, the ecto domain of the BMP receptor protein BMRIA, or other appropriate antagonists of BMP. In one embodiment, the BMP antagonist includes noggin. In another embodiment, the activator or inhibitor can be TGF-β signaling pathway. In one embodiment, the secreted molecule of the TGF-β pathway can include, but is not limited to, nodal.

The invention provides in another aspect, pharmaceutical compositions including therapeutically effective amounts of any of the cell compositions described herein and a pharmaceutically acceptable diluent, excipient, or carrier.

The carrier(s) must be “acceptable” in the sense of being compatible with the other ingredients of the formulation and not deleterious to the recipient thereof. The formulations include those suitable for ophthalmic administration. The most suitable route may depend upon the condition and disorder of the recipient. The formulations may conveniently be presented in unit dosage form and may be prepared by any of the methods well known in the art of pharmacy.

Formulation and Administration

Formulations of the present invention suitable for administration may be presented as a solution or a suspension in an aqueous liquid or a non-aqueous liquid; or as an oil-in-water liquid emulsion or a water-in-oil liquid emulsion. The active ingredient may also be presented as a bolus, electuary or paste.

The pharmaceutical compositions may include a pharmaceutically acceptable inert carrier, and this expression is intended to include one or more inert excipients, which include starches, polyols, granulating agents, microcrystalline cellulose, diluents, lubricants, binders, disintegrating agents, and the like. “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

Examples of excipients for use as the pharmaceutically acceptable carriers and the pharmaceutically acceptable inert carriers and the aforementioned additional ingredients include, but are not limited to:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof;

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof;

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof;

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Degussa Co., Plano, Tex. USA), a pyrogenic silicon dioxide (CAB-O-SIL, Cabot Co., Boston, Mass. USA), or mixtures thereof;

ANTI-CAKING AGENTS: calcium silicate, magnesium silicate, silicon dioxide, colloidal silicon dioxide, talc, or mixtures thereof;

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof; and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof.

Making of Pharmaceutical Preparations: The cells used in the compositions of the present disclosure will typically be cultured and formulated in accordance with methods that are standard in the art. The cells may be prepared in admixture with conventional excipients, carriers, buffers, flavoring agents, etc. Typical carriers include, but are not limited to: water; culture medium; salt solutions; alcohols; gum arabic; vegetable oils; benzyl alcohols; polyethylene glycols; gelatin; carbohydrates, such as lactose, amylose or starch; magnesium stearate; talc; silicic acid; paraffin; perfume oil; fatty acid esters; hydroxymethylcellulose; polyvinyl pyrrolidone; etc. Pharmaceutical preparations can be sterilized and, if desired, mixed with auxiliary agents such as: lubricants; preservatives; disintegrants; stabilizers such as cyclodextrans; wetting agents; emulsifiers; salts; buffers; natural or artificial coloring agents; natural or artificial flavoring agents; or aromatic substances. Pharmaceutical preparations can also include one or more of the following: acetylated monoglyceride, aspartame, beta carotene, calcium stearate, carnauba wax, cellulose acetate phthalate, citric acid, citric acid anhydrous, colloidal silicon dioxide, confectioner's sugar, crospovidone, docusate sodium, ethyl alcohol, ferric oxide, fructose, gelatin, glycerine, glyceryl monostearate (e.g. glyceryl monostearate 40-50), glyceryl triacetate, HPMC (hydroxypropyl methylcellulose), hydroxypropyl cellulose, hypromellose, iron oxide, isopropyl alcohol, lactose monohydrate, low substituted hydroxypropyl cellulose, magnesium carbonate, magnesium stearate, maltol, mannitol, methacrylic acid, methacrylic acid copolymer (e.g. methacrylic acid copolymer type C), methylcellulose, microcrystalline cellulose, mono ammonium glycyrrhizinate, n-butyl alcohol, paraffin, pectin propylene glycol alginate, polyacrylate, polyethylene glycol (e.g. polyethylene glycol 6000), polysorbate 80, polyvinyl pyrrolidone, povidone, propylene glycol, shellac, silicon dioxide, sodium carbonate, sodium citrate, sodium hydroxide, sodium lauryl sulfate, sodium stearyl fumarate, sorbitol, starch, sucrose, sugar sphere, talc, titanium dioxide, triethyl citrate, and xanthan gum.

A variety of administration routes can be used in accordance with the present disclosure. For example, an effective amount of the composition described herein can be administered by any appropriate means of injection and/or implantation. Some appropriate forms of injection may include intracameral injection, intravitreal injection, intracanicular injection, subconjunctival injection, posterior chamber lens injection, and intraocular lens implantation. Administration by implantation may include, for example, encapsulating the cell compositions within a bioartificial organ (such as disclosed by U.S. Pat. No. 5,795,790, the teachings of which are incorporated herein by reference) or within an implantable capsule (such as disclosed by U.S. Pat. No. 5,904,144, the teachings of which are incorporated herein by reference) and implanting these devices in the eye. Booster injections or additional implantations can be performed as required.

In certain embodiments, formulations of the compositions described herein may further include one or more other biological factors and/or agents that influence or direct cell proliferation and/or differentiation. For example, certain extrinsic and intrinsic factors can regulate or bias the differentiation of retinal stem cells to rod photoreceptors. Such extrinsic and intrinsic regulators of rod photoreceptor differentiation include, for example, VEGF, retinoic acid, taurine, Ihh, Activin, IGF-1, FGF-2, S-laminin, Crx (also named Otx5 or Otx5b), NeuroD, Xngnr-1, Ath3 and Otx2. Such biological factors and/or agents can be used prior to or concomitant with injection and/or implantation of the compositions described herein. In this manner the reprogrammed cells (now effectively retinal stem cells) can be partially differentiated into one or more retinal cell type that is desirous of being repopulated. As used herein the term “partially differentiated” refers to cells that are specified to form a retinal cell type but have not yet begun to express all differentiated cell markers.

Other biological factors and/or agents that influence or direct retinal stem cell proliferation and/or differentiation include, for example, FGF-1, EGF, SCF, IGF-II, insulin, Notch, LIF, CNTF, TGF-α, TGF-β-3, Shh, Ath5, Brn3, Ngn2, thyroid hormone, Chx10, Ash1 p27^{Xic1}, NT-3, among others.

Dosing and Regimen

Doses of the aforementioned cell compositions can be suitably decided depending on the purpose of administration, i.e., therapeutic or preventive treatment, nature of a disease to be treated or prevented, conditions, body weight, age, sexuality and the like of a patient. In the method for administering the pharmaceutical preparation according to the present disclosure, the cell compositions may be administered simultaneously with one or more biological factors and/or agents that influences or directs the reprogrammed cells toward proliferation and/or differentiation, or the two may be sequentially administered in an optional order. The practically desirable method and sequence for administration varies depending on the purpose of administration, i.e., therapeutic or preventive treatment, nature of a disease to be treated or prevented, conditions, body weight, age, sexuality and the like of a patient. The optimum method and sequence for administration of the compounds described in detail herein under preset given conditions may be suitably selected by those skilled in the art with the aid of the routine technique and the information contained in the present specification and field of invention. In certain embodiments, an amount of about 10,000-20,000 cells can be administered via a single direct injection or implantation.

Methods of the Invention and Agents Useful Therein

In a further aspect, the invention provides methods of treating and preventing visual impairment by administering a therapeutically effective amount of the compositions described herein to a subject in need thereof. In one embodiment, the administering step is performed by direct injection of the composition into the eye of the subject. In another embodiment, the administering step is performed by implantation of the composition into the eye. In still another embodiment, the administering step is performed using both injection and implantation.

For the purpose of this application, the term “visual impairment” is broadly defined to include any limitation of visual capability which may lead to partially sighted vision or more significant loss of vision, or even blindness. Such visual impairment may include any vision loss that may or may not be related to disease or illness. Visual impairment may be caused by, for example, glaucoma, retinitis pigmentosa, age-related macular degeneration, diabetic retinopathy, retinal injuries, retinal degeneration, albinism, cataracts, muscular problems that result in visual disturbances, corneal disorders, congenital disorders, infections caused by the brain or nervous system, and visual loss due to trauma or injury. Accordingly, the compositions described herein are intended to treat or prevent these disorders.

The terms “treating” or “preventing” mean amelioration, prevention or relief from the symptoms and/or effects associated with the particular visual impairment disorder. The term “preventing” as used herein refers to administering a medicament beforehand to forestall or obtund an acute episode or, in the case of a chronic condition to diminish the likelihood or seriousness of the condition. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis.

As used herein, “administering” or “administration of” a drug or pharmaceutical composition or formulation described herein to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to a subject in need thereof.

As used herein, a “therapeutically effective amount” of a drug or pharmaceutical composition or formulation, or agent, described herein is an amount of a composition that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

In yet another aspect, the invention provides methods for reprogramming a population of non-retinal cells to retinal stem cells by first providing a cell population including one or more non-retinal cell types, and then genetically altering the cells to express or over-express a gene set including an eye-field transcription factor cocktail, thereby reprogramming the non-retinal cells to form retinal stem cells.

As used herein, the phrase “genetically alter” refers to the introduction of one or more exogenous polynucleotide sequences into a cell. The sequences may be duplicates of sequences already in the cell's genetic material as might be the case where over expression is the goal. Or, the sequences may be entirely exogenous, such as would be the case if the cell does not normally express the factor encoded by the sequence.

As would be understood by one of ordinary skill in the art to which the invention pertains, the term “express or over-express” means that although some eye-field transcription factors may be naturally expressed by the non-retinal cells, these cells can be genetically altered to over express the factors in order to successfully reprogram the cells. If, on the other hand, a desired factor is not naturally expressed, the cells can likewise be genetically altered to express it.

Nucleic acid expression constructs for genetically altering non-retinal cell types for use in the methods herein can be constructed by routine methods known to those of skill in the art. As used herein, a “nucleic acid expression construct” refers to an artificially constructed segment of nucleic acid that is going to be transplanted into a target tissue or cell. Preferably the construct contains one or more DNA inserts, which contains the gene sequence encoding one or more of the EFTFs, that has been subcloned into a vector. The vector can contain bacterial resistance genes for growth in bacteria, and promoters for expression in the organism. In a presently preferred embodiment, the construct includes one or more promoter sequences for directing the expression of the EFTF inserts. As known to those skilled in the art, a “promoter” is a DNA sequence that facilitates the binding of RNA polymerase to a template and initiates replication. A promoter initiates transcription only of the gene or genes physically connected to it on the same stretch of DNA, that is, the promoter must be “in cis” with the gene it affects. A promoter may be constitutive, that is, always “on” and capable of initiating transcription at any time. It may be tissue specific and only initiate transcription in certain tissue environs. Or it may be inducible, in which case another molecule, known as an effector, or some other external influence such as, without limitation, temperature, light, shear stress, pH, pressure, etc., is needed to “induce” the promoter to operate. Any of these types of promoters may be used in the constructs of this invention and are within its scope.

In one embodiment, the non-retinal cell types are ectodermal cells. In another embodiment, the ectodermal cells can be epidermal stem cells. In still another embodiment, the non-retinal cells can be embryonic stem cells that are converted to ectodermal cells and then to retinal stem cells. In yet another embodiment, the eye-field transcription factor cocktail includes, but is not limited to, one or more nucleic acid sequences encoding Otx2, ET, Rx1, Pax6, Six3, tll, Optx2, and other sequences, or orthologs thereof.

In another aspect, the invention provides methods of reprogramming a population of non-retinal cells, including providing a cell population having one or more non-retinal cell types and exposing the cells to at least one secreted activator or inhibitor of a signaling pathway involved in retinal stem cell formation.

In one embodiment, the signaling pathway is selected from at least one of hedgehog (Hh), wingless (Wnt), transforming growth factor-β (TGF-β), bone morphogenic protein (BMP), insulin growth factor (IGF), and fibroblast growth factor (FGF). In a preferred embodiment, the signaling pathway is BMP. In another embodiment of the invention, the secreted activator or inhibitor of the BMP signaling pathway involved in retinal stem cell formation is an antagonist of BMP. In still another embodiment, the antagonist of BMP can include, for example, one or more of fetuin, noggin, chordin, gremlin, follistatin, Cerberus, amnionless, DAN, and the ecto domain of the BMP receptor protein BMRIA. In a preferred embodiment, the BMP antagonist is noggin.

In another embodiment, the signaling pathway is TGF-β and the secreted molecule is nodal.

In one embodiment, the non-retinal cells are ectodermal cells. In another embodiment, the ectodermal cells can include epidermal stem cells. In still another embodiment, the non-retinal cells are embryonic stem cells that are converted to epidermal stem cells and then to retinal stem cells.

### Materials and Methods

Preparation of RNA: Complementary RNA was synthesized using the Message Machine kit (Ambion, Austin, Tex.) and the linearized plasmid DNA template. Each template plasmid DNA was cut with a unique restriction enzyme to cleave the cDNA at the 3′ end of the transcript, after the SV40 poly-A signal sequence. Not I enzyme was used to cut plasmids GFP (pCS2.GFP; GenBank Accession No.: U76561), XRx1 (pCS2+.XRx1; GenBank Accession No. AF017273.1), also known as RAX (GenBank Accession No.: AAH51901) in mammals; Xtailless (pCS2+mt.X-tll; GenBank Accession No.: U67886), also known as TLX/NR2E1 in mammals (GenBank Accession No.: NM_{—}152229), XET (pCS2R.XET; GenBank Accession No.: AF173940) also known as TBX2 in mammals (GenBank Accession No.: U28049), XPax6 (pCS2R.XPax6; GenBank Accession No: U76386), XOtx2 (pCS2.XOtx2; GenBank Accession No.: Z46972), XOptx2 (pCS2.XOptx2; GenBank Accession No.: AF081352), also known as SIX6 in mammals (GenBank Accession No.: NM_{—}007374) and Xnoggin (pCS2.Xnoggin; GenBank Accession No.: U16800 and U16801; human noggin GenBank Accession No.: U31202) while XSix3 (pCS2R.XSix3; GenBank Accession No.: AF167980) also known as SIX3 in mammals (GenBank Accession No.: NM_{—}011381 was cut with Pvu II. These clones are all cDNAs from Xenopus laevis (except GFP) cloned into the expression vector, pCS2+ or pCS2R (pCS2+ vector with a repaired T7 sequence site). We followed the protocol for RNA synthesis, using the Phenol/Chloroform method of purification without treating our samples with DNase. After determining our concentration, we resuspend our RNAs in nuclease-free water (Ambion) and store aliquots in the −80° C. freezer.

Preparation of Embryos for microinjection. The female and male frog, Xenopus laevis, are used to produce embryos for RNA blastomere injection. Oocytes are collected from hormonally induced female frogs using a standard X. laevis egg laying procedure: injecting frogs in the dorsal lymph sac first, with pregnant mare serum gonadotropin (200 units) then, 3 to 5 days later with human chorionic gonadotropin (500 units). To collect the eggs, frogs are placed in low saline water and allowed to naturally lay their eggs. The testes are collected from the males, which are anaesthetized by tricaine or by cold, and then decapitated. To fertilize the eggs, oocytes are collected into a 60 mm Petri dish and washed twice in 1×MMR (Marc's Modified Ringer's solution; 10×MMR=1 M NaCl; 20 mM KCl; 10 mM MgCl2; 20 mM CaCl2; 50 mM HEPES, pH 7.5). The testes are macerated in a 1.5 ml tube with 1×MMR. After the 1×MMR solution is removed from the eggs, the resuspended testes is dropped onto the eggs and they are stirred together. After two minutes, 0.1×MMR is poured onto the eggs to cover them and they are left to develop without perturbation. One hour later, the jelly coats of the embryos are removed. To do this, the 0.1×MMR solution is removed and replaced with the dejelly solution [0.2M Tris pH 8.8+3.3 mM DTT (Dithiothreitol; SIGMA Aldrich Inc., St. Louis, Mo.)]. The embryos are allowed to incubate in this solution until we notice the coat has dissolved. They are washed in 0.1×MMR 5-6 times before they are placed into injection dishes containing 0.4×MMR+6% Ficoll.

Injection of Xenopus embryos: To inject embryos, they are placed in 60 mm Petri dishes containing 1% agarose molds with the 0.4×MMR+6% Ficoll solution. The molds have 100 round bottom wells measuring 1.5 mm diameter, just the right size to hold an early developing X. laevis embryo. GFP and/or EFTF RNAs are injected into both blastomeres of two-cell stage embryos and allowed to grow in 0.4×MMR & 6% ficoll at 14° C. overnight. Embryos were injected with 500 picograms (pg) of GFP-only or GFP plus noggin (50 pg) or the following amounts of each EFTF RNA in the EFTF-cocktail (in units of picograms per blastomere): Otx2, 37.4; ET, 75.2; Rx1, 74.9; Pax6 150.2; Six3, 37.4; tll, 37.6, Optx2, 37.6 or noggin alone 50.

Isolation of primitive ectoderm from Xenopus embryos and treatment with Noggin protein: When the embryos reach stage 9, they are transferred to 0.7×MMR & gentamicin (50 μg/ml) solution in a surgical petri dish, which has 100 round-bottomed 1.5 mm wells of 1% agarose+0.7×MMR. Animal caps (ectoderm) are removed using a Gastromaster equipped with a 13 μm microsurgery tip, bent to a width of 400 μm (Xenotek Engineering, Belleville, Ill.). Caps are cultured at 14° C. to stage 15 and serve as donor tissue. As used herein, the term “animal cap” refers to primitive ectoderm isolated from a stage 9 Xenopus laevis embryo previously injected at the two cell stage with the EFTF cocktail (and the fluorescent tracer GFP). The term “noggin cap” as used herein refers to primitive ectoderm isolated from a stage 9 Xenopus laevis embryo previously injected at the two cell stage with noggin RNA (and the fluorescent tracer GFP). “Noggin caps” have also been made by soaking the freshly isolated GFP expressing ectoderm at stage 9 in 1 μM concentration of Noggin/Fc protein (SIGMA, catalog# N6784) in 0.1× phosphate buffered saline+0.02% bovine serum albumin. The externally treated cap tissue remains in this solution until sibling embryos reach stage 15. Similarly, the term “GFP cap” as used herein refers to primitive ectoderm isolated from a stage 9 Xenopus laevis embryo previously injected at the two cell stage with the fluorescent tracer GFP.

Removal of host Xenopus embryo eye primordia and transplantation of EFTF-expressing primitive ectoderm to host embryos: Stage 15 (host) embryos are placed in a surgical dish in 0.7×MMR & gentamicin, the vitellin membrane is removed using #5 forceps and one eye field is removed using the Gastromaster with a 13 micron tip, bent to a width of 200 μm. The donor animal cap is cut in half and placed in the surgical hole. Rotation of the embryo into the well wall or a glass coverslip fragment ensures the tissue remains in place. Embryos are allowed to heal overnight at 18° C. The next day, GFP-positive host embryos are identified using a fluorescent dissecting microscope. Typically, 95-100% of the embryos are GFP-positive. The embryos are transferred into petri dishes and grown in 0.1×MMR at 18° C. until stage 41-43 at which time, they are processed for analysis.

Analysis of embryo phenotypes using in situ hybridization, immunocytochemistry, BrdU labeling and electroretinography: In situ hybridization and immunocytochemistry were done as previously described (Zuber et al., 2003). To perform BrdU labeling, anesthetized stage 43, EFTF-cap containing embryos were placed in 0.7×MMR+gentamicin (50 μg/ml) and injected with ˜30 nl BrdU (10 mM) into the gut. The embryos were fixed in 4% PFA/1×PBS after 1 hr, sunk in 20% sucrose, mounted in O.C.T. and cryostat sectioned. Sections were stained using an anti-BrdU primary antibody (Roche Applied Science, Indianapolis, Ind.) and a 1:500 dilution of Cy3-conjugated goat anti-mouse secondary antibody (Chemicon International, Inc., Temecula, Calif.). Electroretinograms (“ERGs”) were performed as follows: Traces were recorded in response to brief flashes (20 ms) of green light (520 nm). The magnitude of the b-wave was a function of light intensity, saturating and well fit to a Michaelis-Menten function with EC₅₀=220 photons/μm². The Committee for the Humane Use of Animals at SUNY Upstate Medical University approved all protocols.

Various patent and/or scientific literature references have been referred to throughout the instant specification. The disclosures of these publications in their entireties are hereby incorporated by reference as if completely written herein. In view of the detailed description of the invention, one of ordinary skill in the art will be able to practice the invention as claimed without undue experimentation. The foregoing will be better understood with reference to the following Examples that detail certain procedures for making and using the invention. The following Examples should not be considered exhaustive or to limit the scope of the invention, which is defined by the appended claims. Rather, the Examples are merely illustrative of a few of the many embodiments contemplated by the present disclosure. Other aspects, advantages, and modifications are within the scope of the following claims as will be apparent to those skilled in the art.

### EXAMPLES

### Example 1

### EFTFs Reprogram Primitive Ectoderm to Eyes

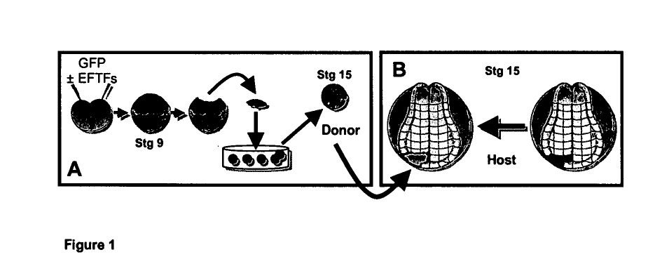

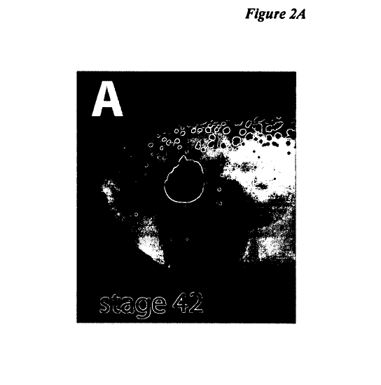

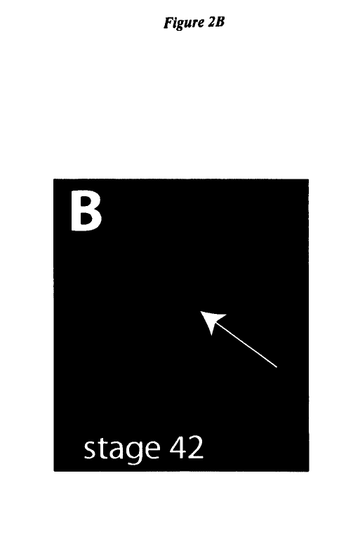

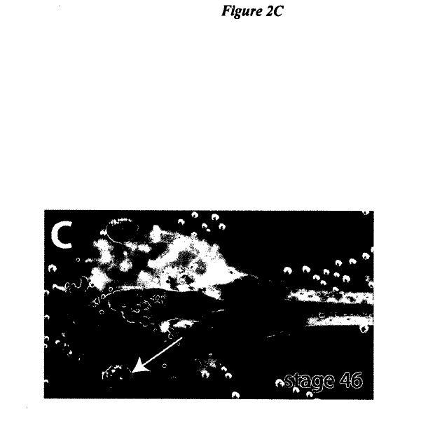

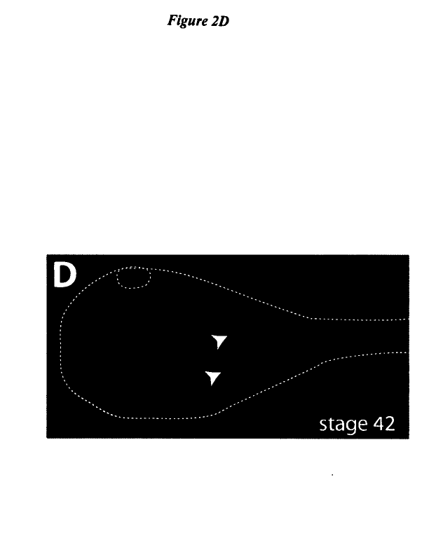

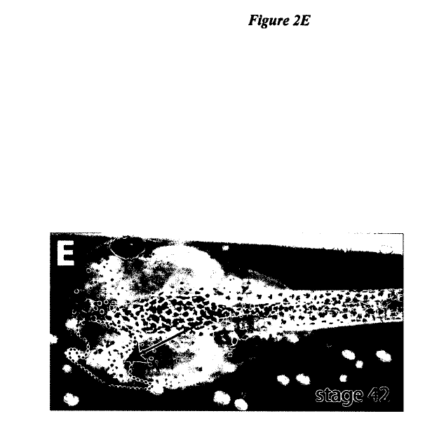

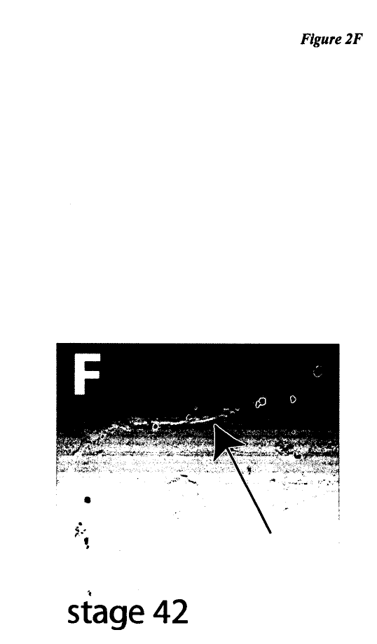

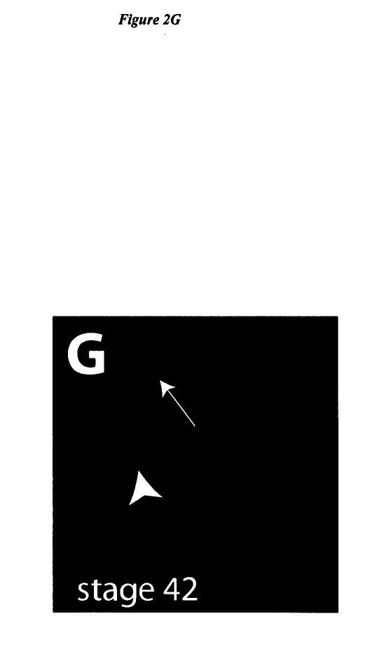

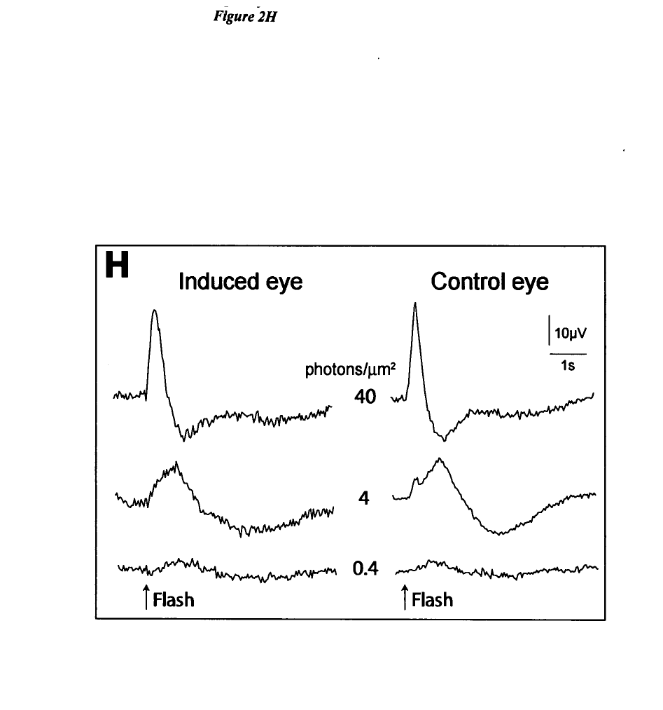

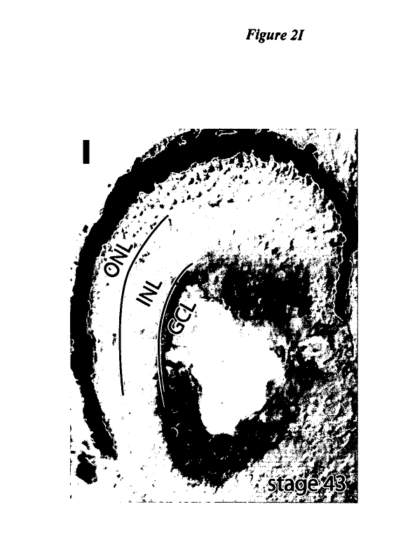

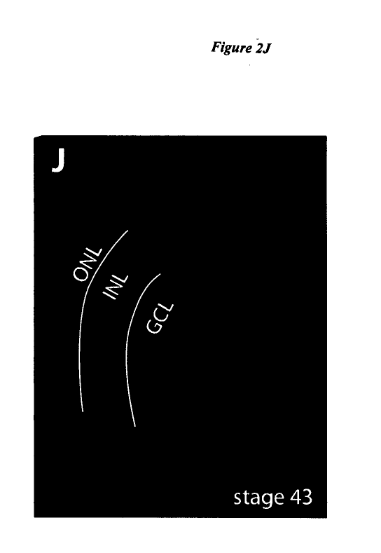

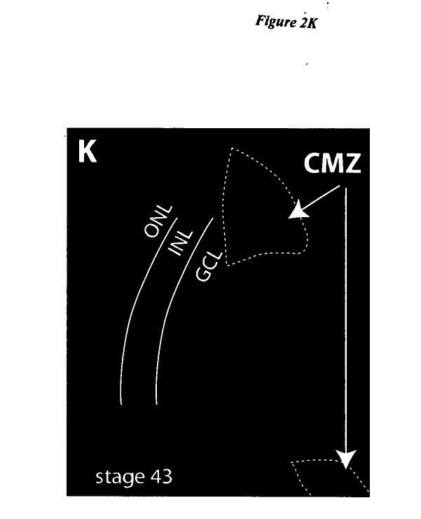

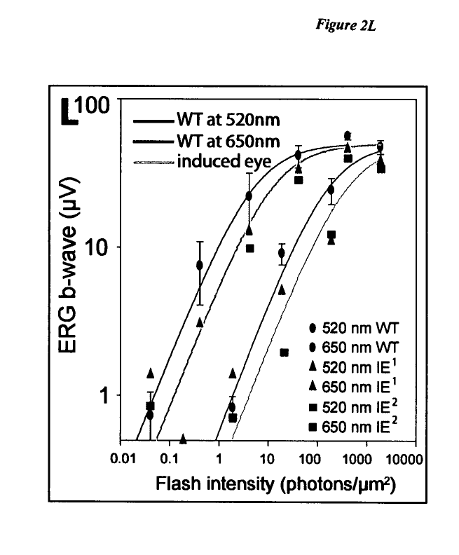

Seven eye field transcription factors (EFTFs) that are expressed in the retinal stem/progenitor cells of the early eye primordia are sufficient to induce the formation of ectopic eyes. An Animal Cap Transplant (ACT) assay makes it possible to detect the formation of retinal stem/progenitor cells. This method makes it possible to determine if non-retinal cells have been reprogrammed to retinal stem/progenitor cells based on their unique ability to generate retinal tissue when transplanted to the developing Xenopus embryo. The ACT assay is schematized in FIG. 1 and a description is detailed in Methods. This assay takes advantage of two strengths of the Xenopus system—the ectodermal explant assay and tissue transplantation assays. Both blastomeres of two-cell stage Xenopus embryos were injected with either EFTF RNA cocktail containing GFP RNA as a tracer or GFP RNA alone. Ectodermal explants (animal caps) are collected from injected embryos and grown in culture until sibling embryos reach stage 15 at which point the tissue was transplanted to host animals from which one eye primordia had been removed. The embryos were then grown to later developmental stages for analysis. FIG. 2 shows results from representative experiments in which GFP (tracer only) and GFP+EFTFs were expressed in primitive ectoderm and the ACT assay performed. Control (GFP-caps) never (n=107 transplants in 5 independent experiments) form eye tissue, primitive ectoderm maintains its normal fate and generates skin epidermis (FIG. 2D-G). This is most clearly demonstrated in sectioned embryos. GFP fluorescence is only detected in the skin (FIGS. 2F & G).

Primitive ectoderm expressing EFTFs (EFTF-caps) formed eyes with external morphology identical to normal tadpole eyes (FIG. 2A-C). On average, 61% of EFTF-cap transplants form eye tissue (57 of 93 transplants in 5 independent experiments). All eyes that formed from EFTF-cap transplants expressed GFP (57/57 transplants) demonstrating they originated from the donor, transplanted, tissue (EFTF-cap). Although some variability in the size of the EFTF-induced eye (i.e., the eye that forms from primitive ectoderm as determined in the ACT assay) was observed, by stage 47 the EFTF-induced eye was approximately equal in size to the eye on the unoperated side of the embryo. Induced eyes also contained a lens and darkly pigmented RPE (FIGS. 2A-C).

### Example 2

### EFTF-Induced Eyes are Morphologically and Molecularly Identical to Normal Eyes

To better characterize the internal morphology and identify cell types present in EFTF-induced eyes, embryos with strongly fluorescent EFTF-induced eyes were fixed, cryostat sectioned and in situ hybridization or immunocytochemistry were used to identify retinal cell types. Induced eyes had internal morphology identical to normal eyes, containing the tri-layered structure of a normal retina and all the cell types that could be identified by morphology and available molecular markers. These including a lens, retinal pigment epithelium (RPE), rod and cone photoreceptors, and retinal ganglion cells (FIG. 2I-J). Retinal ganglion cell (RGC) axons, the only neural processes that leave the retina, exit the back of the eye as the optic nerve. When viewed using high contrast microscopy, axon tracts were observed exiting the back of induced eyes (opposite the lens), reminiscent of the path taken by RGC axons (not shown).

In both fish and amphibians, the retina contains a population of self-renewing adult retinal stem cells. This ciliary marginal zone or CMZ is located in the periphery of the eye and contains a slowly proliferating population of cells that differentiate into new retinal cells throughout the life of the animal. To determine if the EFTF-induced eye contained this population of stem cells, we injected the thymidine analog 5-bromo-2-deoxyuridine (BrdU) into the gut of tadpoles that had developed EFTF-induced eyes. BrdU, is incorporated in the DNA of cycling cells and its presence can be detected using BrdU specific antibodies (see methods). BrdU immunoreactivity was detected in the peripheral retina consistent with the position of the adult retinal stem cells of the CMZ. EFTF-cap cells form eyes with the internal morphology and every cell type that could be detected using available molecular markers.

### Example 3

### EFTF-Induced Eyes are Functionally Normal

In vertebrate eyes, the cornea and lens focus light reflected from images in the surrounding world onto the retina, which lines the back of the eyeball. Cells in the retina form complex circuits designed to convert light into electrical impulses that pass via RGC axons to the brain. An electroretinogram (ERG) can 1) detect additional retinal cell types not identifiable using molecular markers, 2) determine if the induced cells were functionally normal and 3) determine if they formed the intricate neural network necessary to detect and process a light stimulus. EFTF-induced eyes generated ERGs typical of normal eyes (FIGS. 2H & L). In the outer retinal layer, rod and cone photoreceptors use phototransduction to convert light into an electrical impulse. In the normal retina, photoreceptor initiated impulses pass through the inner nuclear layer via second order cell types. In induced eyes, brief light flashes with intensities as low as 0.4 photons/μm² generated a positive b-wave (FIG. 2H). Inner nuclear layer cells post-synaptic to the photoreceptors drive the b-wave, which is due primarily to On-bipolar cells. The magnitude of the b-wave increased with flash intensity and saturated in response to light intensities of 100 to 1000 photons/μm², depending on the wavelength of illumination used (FIG. 2H). The ERG requires the sequential activity of multiple retinal cell types. Disruption in any part of the system would result in an abnormal or no detectable ERG. ERG traces from EFTF-induced and control eyes are virtually identical in every respect. Therefore, the recordings from ectopic eyes not only indicates the presence of functional photoreceptors, bipolar cells, and the retinal pigment epithelium, but also demonstrates; that light enters the eye appropriately, the intracellular signal transduction pathways (phototransduction, etc.) within each cell type are active, that synapses form between cells, and that synaptic transmission is normal. Engineered retinal stem/progenitor cells are multipotent and self-renewing as they differentiate into every cell type necessary to form a functional eye—including the adult retinal stem cell of the ciliary marginal zone.

### Example 4

### The Secreted Polypeptide Noggin Mimics the Ability of EFTFs to Reprogram Primitive Ectoderm to Eyes

Despite the remarkable ability of EFTF-caps to form eyes that are anatomically, molecularly and functionally indistinguishable from the endogenous eye, a similar approach to transforming cultured pleuripotent non-retinal mammalian (including human) cells to retinal stem/progenitor cells is challenging as such an approach would require that the cells to be reprogrammed were expressing each EFTF under the control of inducible promoters that would allow for coordinated and tightly regulated expression of each EFTF at the level necessary to specifically reprogram mammalian embryonic stem cells (human or mouse for example) to retinal stem/progenitor cells.

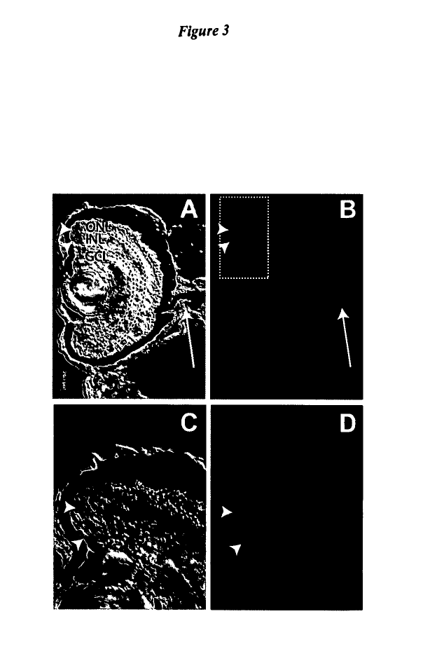

An alternative approach is to identify extrinsic factors to accomplish this same result as that of the EFTF-induced eye. The secreted neural inducer noggin can activate the expression of EFTFs in primitive ectoderm. Noggin is a soluble protein, which acts via its ability to inhibit BMP signaling. To determine if noggin functionally replaces the EFTF cocktail, noggin protein is expressed in primitive ectoderm and the ACT assay in Xenopus embryos is performed. FIG. 3 shows a typical result when primitive ectoderm expressing noggin protein is transplanted to host embryos. GFP expression (transplanted tissue) is observed throughout the retina. Cells in all three nuclear layers express GFP, indicating that all retinal cell types can be generated from primitive ectoderm once they have been reprogrammed to retinal stem/progenitor cells by noggin protein. One hundred percent (100%, n=13) of embryos receiving noggin-cap transplants contain GFP expressing eyes. This result was repeated with caps simply treated with commercially available Noggin protein. In contrast, no Xenopus embryos receiving GFP-caps formed eyes. Therefore, consistent with our previous molecular analysis, which demonstrated that noggin induced the expression of the EFTFs in primitive ectoderm, noggin also mimics the ability of the EFTFs to reprogram primitive ectoderm to retinal stem/progenitor cells.

### Example 5

### Reprogramming Non-Retinal Cells to Retinal Stem/Progenitor Cells In Vitro

Seven eye field transcription factors (EFTFs) are expressed in the retinal stem/progenitor cells of the early eye primordia and are sufficient to induce the formation of ectopic eyes in vivo. This same cocktail can be used to reprogram pluripotent ectoderm to retinal stem/progenitor cells in culture. When one of the two endogenous eye fields is replaced with EFTF-expressing cells, the transplanted tissue forms a complete eye that is anatomically and functionally indistinguishable from the normal eye—including the presence of adult retinal stem cells. Artificially generated vertebrate retinal cells can be created in culture and these cells, when reintroduced into the animal, form an eye with all the neural circuitry necessary to respond normally to a light stimulus. Reprogramming of primitive ectoderm to retinal stem/progenitor cells can also be accomplished (with even higher efficiency) using the secreted polypeptide noggin. These results demonstrate that noggin (and other secreted factors possessing similar activities) can be used to reprogram cultured mammalian (human and mouse) pleuripotent, non-retinal cell types such as embryonic stem cells and stem cells of other lineages that can be isolated from a patient's own tissues.

### Example 6

### Conversion of Pleuripotent Mammalian Stem Cells (Embryonic Stem Cells and Adult Stem Cells) Isolated from Animals and Patients

Transplantation results with noggin demonstrate that diffusible factors can substitute for the EFTFs and reprogram pleuripotent, non-retinal cell types to retinal stem/progenitor cells in the amphibian Xenopus laevis. Despite dramatic differences in developmental time scale and size, human and frog retinas share similarities in basic structure, function and development. For example, all seven major retinal cell classes seen in humans are also found in the frog eye. The retinas of both species are organized into three distinct cellular layers. In addition to structural similarities, homologous, retinal-specific genes are required for the normal development of the eye in both species. Thus, those of ordinary skill in the art will acknowledge that the studies and findings in Xenopus are reasonably correlative and predictive of what will occur in other vertebrates, including humans.

As demonstrated above, an ideal source of cells for the in vitro generation of retinal stem/progenitor cells is primitive ectoderm. This tissue source is remarkable in its ability to respond to extrinsic factors (noggin in the above examples) and form retinal stem/progenitor cells. Using the techniques provided by Rathjen et al. Methods of Enzymology Review (2004), embryonic stem cells are converted into a nearly pure early primitive ectoderm-like lineage (EPL). Using the methods described herein, these EPLs can then be directed to multipotent, retinal stem cell lineage for use in cell replacement therapies for degenerated or damaged adult retina.

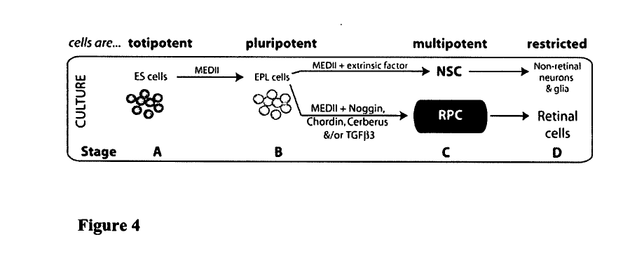

FIG. 4 and the following paragraph below describes in brief how mouse ES cells, for instance, can be reprogrammed and purified to generate a relatively homogeneous population of retinal stem/progenitor cells. Those of ordinary skill in the art would understand and recognize that this same protocol can be used to convert other pleuripotent, non-retinal cell types (originating from both human and mouse cells) to retinal stem/progenitor cells.

Briefly, mouse ES cells harbouring green fluorescent protein (GFP) under the control of the retinal progenitor-specific region of the mouse Pax6 promoter (RetPax6->GFP) are generated. ES cells that are successfully converted to retinal progenitors express GFP and can therefore be quantitated and purified by fluorescence activated cell sorting (FACS). A similar approach was used to isolate and characterize neuroectoderm progenitors expressing GFP under the control of the mouse Sox1 promoter. Mouse ES cells containing the RetPax6->GFP transgene are cultured in MEDII media. MEDII media is sufficient to convert greater than 96% of ES cells to EPL cells. Like ES cells, EPL cells can be continuously cultured, but unlike ES cells, EPL cells can be directed to a virtually pure neuroectodermal cell lineage. Until now a homogeneous culture of ungenetically modified primitive neuroectoderm has not been available anywhere and thus, this approach was not possible. EPL cells can then be biased toward a retinal lineage, using noggin in combination with other extrinsic factors (e.g., chordin, cerberus and TGF-β3) known to restrict primitive ectoderm and neuroectoderm towards a retinal stem/progenitor cell fate. Thus, using these methods it is possible to reprogram cultured pleuripotent, non-retinal human or mouse cells to retinal stem/progenitor cells.

### Example 7

### Vision-Based Behavioral Assay

Although the ERG can be used to test that non-retinal cells reprogrammed to retinal stem cells and eventually retinal cells are functional, it has limitations in that it cannot determine whether all the retinal cell types that were formed are functional. For example, it cannot detect all the signaling that must take place for sight. Moreover, it cannot determine whether the RGCs are functioning normally.

In order to overcome these limitations of the ERG assay, a vision-based behavioral assay was used to show that the Xenopus tadpole with EFTF-induced or noggin-induced eyes can in fact see normally.

Briefly, normal Xenopus tadpoles are placed in a tank that is white colored on one side and black colored on the other. The normal tadpole swims to and stays on the white colored side. This behavior is known to be vision-based because if the connection between the eye and brain is severed (effectively blinding the tadpole) the tadpoles do not stay on the white side but spend equal amounts of time on both the white and black side of the tank.

Xenopus tadpoles with EFTF-induced or noggin-induced eyes are placed in tank having white and black colored sides. The uninduced eye/brain connection is severed in these animals. These tadpoles swim to and stay on the white colored side of the tank just as the tadpoles with normal eyes.

### Example 8

### Transplantation of In Vitro Generated Retinal Stem Cells

Retinal stem cells derived from reprogrammed non-retinal cells are generated as described in Examples 5 or 6 and kept in culture conditions that maximize retinal stem cell numbers. The optimum time for transplanting the retinal stem cells is determined by using RT-PCR to detect the expression time course of markers specific for retinal progenitor and differentiating retinal cells, indicating the age of the retinal stem cell.

Cultured retinal progenitor cells are stained with an inert, long-lasting cell-autonomous dye (PKH FLuorescent Cell Linker Dye; SIGMA) and transplanted into neonate, adult wild-type, and rd/rd mouse retinas as described in A. Otani et al. J. Clin Invest 144, 765 (2004) and A. Otani et al., Nat Med 8, 1004 (2002). Then, 10,000-20,000 are injected intravitreally into the mice.

Visual acuity and spatial vision of experimental and sham mice is determined using the ERG and visual optomoter system (VOS). Retinas are sectioned and stained with retinal cell-type specific markers to determine survival, integration, and differentiation of transplanted cells in the host retina, (according to B. L. Coles et al. Proc Natl Acad Sci USA 101, 15772 (2004) and D. M. Chacko et al., Biochem Biophys Res Commun 268, 842 (2000)), thereby determining rescue of the retina at the cellular level and restoration of sight in the living animal.

### REFERENCES

Lighthouse. (2002). Lighthouse International: Statistics on Vision Impairment.

Nei. (2002). National Institutes of Health: National Eye Institute; Vision Problems in the

U.S.—Prevalence of Adult Vision Impairment and Age-Related Eye Diseases in America.

Tropepe, V., Coles, B. L., Chiasson, B. J., Horsford, D. J., Elia, A. J., Mcinnes, R. R., and Van Der Kooy, D. (2000). Retinal stem cells in the adult mammalian eye. Science 287, 2032-2036.

Coles, B. L., Angenieux, B., Inoue, T., Del Rio-Tsonis, K., Spence, J. R., Mcinnes, R., Arsenijevic, Y., and Van Der Kooy, D. (2004). Facile isolation and the characterization of human retinal stem cells. Proc Natl Acad Sci USA 101, 15772-15777.

Lund, R. D., Ono, S. J., Keegan, D. J., and Lawrence, J. M. (2003). Retinal transplantation: progress and problems in clinical application. J Leukoc Biol 74, 151-160.

Ahmad, I., Das, A., James, J., Bhattacharya, S., and Zhao, X. (2004). Neural stem cells in the mammalian eye: types and regulation. Semin Cell Dev Biol 15, 53-62.

Das, A., James, J., Rahnenfuhrer, J., Thoreson, W., Bhattacharya, S., Zhao, X., and Ahmad, I. (2005). Retinal properties and potential of the adult mammalian ciliary epithelium stem cells. Vision Res 45, 1653-1666.

Zuber, M. E., Gestri, G., Viczian, A. S., Barsacchi, G., and Harris, W. A. (2003). Specification of the vertebrate eye by a network of eye field transcription factors. Development 130, 5155-5167.

Gurevich, L., and Slaughter, M. (1993). Comparison of the waveforms of the ON bipolar neuron and the b-wave of the electroretinogram. Vision Res 33, 2431-2435.

Newman, E. (1980). Current source-density analysis of the b-wave of frog retina. J Neurophysiol 43, 1355-1366.

Zimmerman, L. B., De Jesus-Escobar, J. M., and Harland, R. M. (1996). The Spemann organizer signal noggin binds and inactivates bone morphogenetic protein 4. Cell 86, 599-606.

DOWLING J. E. (1987). The Retina: An Approachable Part of the Brain, (Cambridge, Mass.).

Oliver, G., and Gruss, P. (1997). Current views on eye development. Trends Neurosci 20, 415-421.

Jean, D., Ewan, K., and Gruss, P. (1998). Molecular regulators involved in vertebrate eye development. Mech Dev 76, 3-18.

Chow, R. L., and Lang, R. A. (2001). Early eye development in vertebrates. Annu Rev Cell Developmental Biology 17, 255-296.

Klassen, H., Sakaguchi, D., and Young, M. (2004). Stem cells and retinal repair. Prog Retin Eye Res 23, 149-181.

Tabata, Y., Ouchi, Y., Kamiya, H., Manabe, T., Arai, K., and Watanabe, S. (2004). Specification of the Retinal Fate of Mouse Embryonic Stem Cells by Ectopic Expression of Rx/rax, a Homeobox Gene. Mol Cell Biol 24, 4513-4521.

Kammandel, B., Chowdhury, K., Stoykova, A., Aparicio, S., Brenner, S., and Gruss, P. (1999). Distinct cis-essential modules direct the time-space pattern of the Pax6 gene activity. Dev Biol 205, 79-97.

Ying, Q., Stavridis, M., Griffiths, D., Li, M., and Smith, A. (2003). Conversion of embryonic stem cells into neuroectodermal precursors in adherent monoculture. Nat Biotechnol 21, 183-186.

Rathjen, J., Haines, B., Hudson, K., Nesci, A., Dunn, S., and Rathjen, P. (2002). Directed differentiation of pluripotent cells to neural lineages: homogeneous formation and differentiation of a neurectoderm population. Development 129, 2649-2661.

We claim:

1. A composition comprising a population of retinal stem cells, said retinal stem cells comprising a population of reprogrammed non-retinal cells.

2. The composition of claim 1, wherein the population of retinal stem cells is mammalian.

3. The composition of claim 2, wherein said mammalian retinal stem cells are human.

4. The composition of claim 1, wherein said retinal stem cells are capable of producing retinal progenitor cells, retinal cells, and adult retinal stem cells.

5. The composition of claim 4, wherein said retinal cells are selected from one or more of the following: (a) rod cells; (b) cone cells; (c) bipolar cells; (d) amacrine cells; (e) retinal ganglion cells; (f) retinal pigment epithelial cells; (g) Mueller cells; and (h) horizontal cells.

6. The composition of claim 1, wherein the non-retinal cells are selected from ectodermal cells.

7. The composition of claim 6, wherein said ectodermal cells are epidermal stem cells.

8. The composition of claim 7, wherein said epidermal stem cells are reprogrammed embryonic stem cells or cells harvested from a patient's skin, or a combination thereof.

9. The composition of claim 1, wherein the non-retinal cell types are reprogrammed with a gene set comprising an eye-field transcription factor cocktail.

10. The composition of claim 9, wherein the eye-field transcription factor cocktail comprises nucleic acid sequences encoding the following: Otx2; ET; Rx1; Pax6; Six3; tll; Optx2; and orthologs thereof.

11. The composition of claim 1, wherein the non-retinal cell types are reprogrammed by externally applying or causing the cells to express or over express one or more secreted activator or inhibitor of a signaling pathway involved in retinal stem cell formation.

12. The composition of claim 11, wherein said signaling pathway is selected from one or more of the following: (a) hedgehog (Hh); wingless (Wnt); transforming growth factor-β (TGF-β); bone morphogenic protein (BMP); insulin growth factor (IGF); and fibroblast growth factor (FGF).

13. The composition of claim 11, wherein said activator or inhibitor is an antagonist of BMP.

14. The composition of claim 13, wherein said antagonist of BMP is selected from one or more of the following: (a) fetuin; (b) noggin; (c) chordin; (d) gremlin; (e) follistatin; (f) cerberus; (g) amnionless; (h) DAN; and (i) the ecto domain of the BMP receptor protein BMRIA.

15. The composition of claim 14, wherein said antagonist of BMP is noggin.

16. The composition of claim 12, wherein the signaling pathway is TGF-β and the secreted molecule is nodal.

17. A composition comprising a population of non-retinal cell types that have been genetically altered to express or over-express a gene set comprising an eye-field transcription factor cocktail.

18. The composition of claim 17, wherein the eye-field transcription factor cocktail comprises nucleic acid sequences encoding the following: Otx2; ET; Rx1; Pax6; Six3; tll; Optx2; or orthologs thereof.

19. A composition comprising a population of non-retinal cell types that have been externally treated with one or more secreted activator or inhibitor of a signaling pathway involved in retinal stem cell formation.

20. The composition of claim 19, wherein said signaling pathway is selected from one or more of the following: (a) hedgehog (Hh); wingless (Wnt); transforming growth factor-β (TGF-β); bone morphogenic protein (BMP); insulin growth factor (IGF); and fibroblast growth factor (FGF).

21. The composition of claim 19, wherein said activator or inhibitor is an antagonist of BMP.

22. The composition of claim 21, wherein said antagonist of BMP is selected from one or more of the following: (a) fetuin; (b) noggin; (c) chordin; (d) gremlin; (e) follistatin; (f) Cerberus; (g) amnionless; (h) DAN; and (i) the ecto domain of the BMP receptor protein BMRIA.

23. The composition of claim 22, wherein said antagonist of BMP is noggin.

24. The composition of claim 20, wherein the signaling pathway is TGF-β and the secreted molecule is nodal.

25. A pharmaceutical composition comprising a therapeutically effective amount of the composition according to claim 1 and a pharmaceutically acceptable diluent, excipient, or carrier.

26. A pharmaceutical composition comprising a therapeutically effective amount of the composition according to claim 17 and a pharmaceutically acceptable diluent, excipient, or carrier.

27. A pharmaceutical composition comprising a therapeutically effective amount of the composition according to claim 19 and a pharmaceutically acceptable diluent, excipient, or carrier.

28. A method of treating or preventing visual impairment, the method comprising administering a therapeutically effective amount of the pharmaceutical composition of claim 25 to a subject in need thereof.

29. The method of claim 28, wherein said visual impairment is caused by one or more of the following: (a) glaucoma; (b) retinitis pigmentosa; (c) age-related macular degeneration; (d) diabetic retinopathy; and (e) retinal injuries.

30. The method of claim 28, wherein the administering step is performed by injection or implantation.

31. The method of claim 30, wherein the injection is intravitreally.

32. A method of reprogramming a population of non-retinal cells, the method comprising:
(a) providing a cell population comprising one or more non-retinal cell types; and
(b) genetically altering the cells to express or over express a gene set comprising an eye-field transcription factor cocktail,
thereby reprogramming said non-retinal cells to retinal stem cells.

33. A method of claim 32, wherein the non-retinal cells types are ectodermal cells.

34. The method of claim 33, wherein the ectodermal cells are epidermal stem cells.

35. A method of claim 32, wherein the eye-field transcription factor cocktail comprises nucleic acid sequences encoding the following: Otx2; ET; Rx1; Pax6; Six3; tll; Optx2; and orthologues thereof.

36. A method of reprogramming a population of non-retinal cells, the method comprising:
a. providing a cell population comprising one or more non-retinal cell types; and
b. exposing said cells to one or more secreted activator or inhibitor of a signaling pathway involved in retinal stem cell formation.
thereby reprogramming the non-retinal cells to retinal stem cells.

37. The method of claim 36, wherein said non-retinal cells are ectodermal cells.

38. The method of claim 37, wherein the ectodermal cells are epidermal stem cells.

39. The composition of claim 36, wherein said signaling pathway is selected from one or more of the following: (a) hedgehog (Hh); wingless (Wnt); transforming growth factor-β (TGF-β); bone morphogenic protein (BMP); insulin growth factor (IGF); and fibroblast growth factor (FGF).

40. A method of claim 36, wherein said secreted activator or inhibitor of the signaling pathway(s) involved in retinal stem cell formation is an antagonist of BMP.

41. A method of claim 40, wherein said antagonist of BMP is selected from one or more of the following: (a) fetuin; (b) noggin; (c) chordin; (d) gremlin; (e) follistatin; (f) Cerberus; (g) amnionless; (h) DAN; and (i) the ecto domain of the BMP receptor protein BMRIA.

42. A method of claim 41, wherein said antagonist of BMP is noggin.

43. The composition of claim 39, wherein the signaling pathway is TGF-β and the secreted molecule of TGF-β is nodal.

44. A method of reprogramming embryonic stem cells comprising:
(a) providing a cell population of embryonic stem cells;
(b) exposing the embryonic stem cells to factors causing them to differentiate into primitive ectodermal cells; and
(c) exposing the primitive ectodermal cells to one or more secreted activator and inhibitor of a signaling pathway involved in retinal stem cell formation,
thereby reprogramming said embryonic stem cells to retinal stem cells.

45. The composition of claim 44, wherein said signaling pathway is selected from one or more of the following: (a) hedgehog (Hh); wingless (Wnt); transforming growth factor-β (TGF-β); bone morphogenic protein (BMP); insulin growth factor (IGF); and fibroblast growth factor (FGF).

46. A method of claim 44, wherein said secreted activator or inhibitor of the signaling pathway involved in retinal stem cell formation is an antagonist of BMP.

47. A method of claim 46, wherein said antagonist of BMP is selected from one or more of the following: (a) fetuin; (b) noggin; (c) chordin; (d) gremlin; (e) follistatin; (f) Cerberus; (g) amnionless; (h) DAN; and (i) the ecto domain of the BMP receptor protein BMRIA.

48. A method of repopulating one or more retinal cell types, the method comprising:
(a) providing a cell population comprising one or more non-retinal cell types;
(b) exposing the cells to one or more secreted activator or inhibitor of a signaling pathway involved in retinal stem cell formation, thereby reprogramming the non-retinal cells to retinal stem cells; and
(c) injecting the retinal stem cells of step (b) into the retina of a subject in need thereof,
whereby the retinal stem cells differentiate into one or more retinal cell types thereby repopulating one or more retinal cell types that have been damaged or diseased.
